(19) United States
(12) Patent Application Publication
(10) Pub. No.: US 2008/0114303 A1
(43) Pub. Date: May 15, 2008

(54) GUIDEWIRE

(75) Inventor: Anthony Tremaglio, Charlestown, MA (US)

Correspondence Address: GANZ LAW, P.C., P O BOX 2200, HILLSBORO, OR, 97123, US

(73) Assignee: GYRUS ACMI, INC., Southborough, MA (US)

(21) Appl. No.: 11/869,195

(22) Filed: Oct. 9, 2007

Related U.S. Application Data
(60) Provisional application No. 60/828,688, filed on Oct. 9, 2006.

(51) Int. Cl. A61M 25/09

(52) U.S. Cl. 604/164.13

(57) ABSTRACT

A guidewire may include opposing tip portions and two mid-shaft portions adjacent each other and the tip portions. The tip portions each may be relatively less stiff than either mid-shaft portion. The outer cross-sectional profile dimension may be generally uniform across the length of mid-shaft portions. The opposing tip portions may have about the same or smaller cross-sectional profile dimension relative to either mid-shaft portion. The guidewire may have a length and stiffness for each mid-shaft portion that enables the guidewire to be reversibly inserted, with one or the other opposing tip portion leading the way, into a predetermined body passage of a patient. The inventive subject matter is further directed to methods of using a reversible guidewire in a procedure conducted on a patient.

### RELATED APPLICATIONS

This application claims priority to and the benefit of U.S. provisional patent application No. 60/828,688, filed on Oct. 9, 2006, by Anthony Tremaglio, entitled “GUIDEWIRE” the contents of which is hereby incorporated by reference as if recited in full herein for all purposes.

### BACKGROUND

The inventive subject matter generally relates to medical guidewires. More particularly, the inventive subject matter relates to dual floppy guidewires for use in providing a path for endoscopes, access sheaths, catheters, dilators, and stents. In certain particular respects, the guidewires are configured for use in the field of urology.

In addition to urological procedures, guidewires are used in many other procedures in which an endoscope or other instrument needs to be introduced into a lumen. Common examples include vascular and cardiac procedures, thoracoscopic (lung) procedures, neurological procedures, and ENT procedures, such as sinus surgery.

Dual floppy guidewires have a (1) highly lubricious distal tip that allows negotiation of tortuous anatomy; (2) a less lubricious mid-shaft that allows solid handling; and (3) a floppy proximal tail that protects against endoscope damage during backloading. A representative guidewire patent is U.S. Pat. No. 4,925,445, which is hereby incorporated by reference in its entirety.

Existing dual floppy guidewires typically consist of a Nitinol inner core, a polymer jacket tip with hydrophilic coating, a stainless steel jacketed mid-shaft with PTFE coating and a polymer coated tail.

The existing dual floppy guidewires are designed with a uniform stiffness between the floppy tips. If during a procedure the physician needs more or less support than the guidewire offers, a different guidewire must be selected and used, which complicates the procedure and increases its cost. Further, because the length of a given anatomical passage may vary from patient to patient, multiple guidewires may need to be kept available, adding to the complexity and cost of a procedure.

Accordingly there is a need for a single dual floppy guidewire for a medical device that is usable across varying conditions in a medical procedure.

### SUMMARY

The inventive subject matter offers a solution for these problems by providing a reversible dual floppy guidewire with variable stiffness, and methods for using such guidewires. In one possible embodiment, the inventive subject matter provides a guidewire for a medical device with a filamentous structure having a central core including a shape memory material and one or more surrounding coaxial layers; the structure having opposing tip portions and at least two mid-shaft portions interposed between the tip portions, the tip portions each being relatively less stiff than either mid-shaft portion, and the mid-shaft portions having a different stiffness relative to each other; the outer cross-sectional profile dimension being generally uniform across the length of mid-shaft portions, the opposing tip portions having about the same or smaller cross-sectional profile dimension relative to either mid-shaft portion; and wherein the guidewire has a length and stiffness for each mid-shaft portion that enables the guidewire to be reversibly inserted, with one or the other opposing tip portion leading the way, into a predetermined body passage of a patient.

In the foregoing embodiment, the core may have a substantially uniform cross-sectional profile dimension across the length of mid-shaft portions, and the core through the opposing tip portions may have about the same or smaller cross-sectional profile dimension as the core for either mid-shaft portion. In the foregoing embodiment, the core may have a first mid-shaft portion having a different cross-sectional profile dimension than the core of a second mid-shaft portion. In the foregoing embodiment, the thickness of the coaxial layer or layers surrounding the core in a first mid-shaft portion is different from the thickness of the coaxial layer or layers surrounding the core in a second mid-shaft portion. In the foregoing embodiment, the core for a tip portion or mid-shaft portion may have a tapering cross-sectional profile dimension going from one side to another side at least a transition zone between portions. In the foregoing embodiment, the corresponding coaxial layer surrounding the core for the tapering section may have a complementary reverse taper, so that there is an overall cross-sectional profile dimension for the tapering section that is generally uniform. In the foregoing embodiment, there may be a plurality of complementary tapering sections, each with a different stiffness; at least one section of complementary tapering may comprise one opposing tip portion, and another section, the other opposing tip portion, and the core tapers going toward the terminal ends of the tip portions; another section of complementary taper may include a mid-shaft portion. In the foregoing embodiment, the core for a tip portion or mid-shaft portion may have a stepped cross-sectional profile dimension going from one side to another side at least a transition zone between portions. In the foregoing embodiment, the corresponding coaxial layer surrounding the core for the stepped section may have a complementary reverse step, so that there is an overall cross-sectional profile dimension for the stepped section that is generally uniform. In the foregoing embodiment, the coaxial layer or layers may include one or more polymer materials, for example PTFE. In the foregoing embodiment, the opposing tip portions may have end sections comprising a lower durometer polymer coating than the adjacent mid-shaft portions; and/or one opposing tip portion may have a different length relative to the other tip portion.

In another possible embodiment, a guidewire for a medical device, may include a filamentous structure having a central core comprising a super alloy and one or more surrounding coaxial layers; the structure having opposing tip portions and two mid-shaft portions adjacent each other and the tip portions, the tip portions each being relatively less stiff than either mid-shaft portion; the outer cross-sectional profile dimension being generally uniform across the length of mid-shaft portions, the opposing tip portions having about the same or smaller cross-sectional profile dimension relative to either mid-shaft portion; wherein the core for one mid-shaft portion is relatively more flexible than the core for the other mid-shaft portion, and the core sections for each tip portion being about the same flexibility relative to each other; and wherein the guidewire has a length and stiffness for each mid-shaft portion that enables the guidewire to be reversibly inserted, with one or the other opposing tip portion leading the way, into a predetermined body passage of a patient.

In another possible embodiment, a mid-shaft portion may include a core, a first inner coaxial layer may include a stainless steel jacket, and outer coaxial layer may include a polymer coating.

The inventive subject matter also contemplates a method of using reversible dual floppy guidewires. In one possible embodiment of a method, there is a step of providing a guidewire that includes opposing tip portions and two mid-shaft portions adjacent each other and the tip portions, the tip portions each being relatively less stiff than either mid-shaft portion; the outer cross-sectional profile dimension being generally uniform across the length of mid-shaft portions, the opposing tip portions having about the same or smaller cross-sectional profile dimension relative to either mid-shaft portion; and wherein the guidewire has a length and stiffness for each mid-shaft portion that enables the guidewire to be reversibly inserted, with one or the other opposing tip portion leading the way, into a predetermined body passage of a patient. The method further includes steps of inserting the guidewire into a bodily passage of the patient with one of the opposing tip portions leading the way; withdrawing the guidewire and reinserting it with the other opposing tip portion leading the way; and the withdrawal or reinsertion occurring in connection with the placement or removal of a medical instrument over the guidewire, and the instrument's introduction to or from the bodily passage of the patient, in the same treatment episode of treatment.

In another possible embodiment of a method, there is a step of providing a guidewire that includes a filamentous structure having a central core comprising a shape memory material and one or more surrounding coaxial layers; the structure having opposing tip portions and at least two mid-shaft portions interposed between the tip portions, the tip portions each being relatively less stiff than either mid-shaft portion, and the mid-shaft portions having a different stiffness relative to each other; the outer cross-sectional profile dimension being generally uniform across the length of mid-shaft portions, the opposing tip portions having about the same or smaller cross-sectional profile dimension relative to either mid-shaft portion; and wherein the guidewire has a length and stiffness for each mid-shaft portion that enables the guidewire to be reversibly inserted, with one or the other opposing tip portion leading the way, into a predetermined body passage of a patient.

In another possible embodiment of a method, there is a step of providing a guidewire that includes a filamentous structure having a central core comprising a super alloy and one or more surrounding coaxial layers; the structure having opposing tip portions and two mid-shaft portions adjacent each other and the tip portions, the tip portions each being relatively less stiff than either mid-shaft portion; the outer cross-sectional profile dimension being generally uniform across the length of mid-shaft portions, the opposing tip portions having about the same or smaller cross-sectional profile dimension relative to either mid-shaft portion; wherein the core for one mid-shaft portion is relatively more flexible than the core for the other mid-shaft portion, and the core sections for each tip portion being about the same flexibility relative to each other; and wherein the guidewire has a length and stiffness for each mid-shaft portion that enables the guidewire to be reversibly inserted, with one or the other opposing tip portion leading the way, into a predetermined body passage of a patient.

In another possible embodiment, a guidewire for a medical device, may be provided with opposing tip portions and two mid-shaft portions adjacent each other and the tip portions, the tip portions each being relatively less stiff than either mid-shaft portion; the outer cross-sectional profile dimension (e.g., outer diameter for circle) being generally uniform across the length of mid-shaft portions, the opposing tip portions having about the same or smaller cross-sectional profile dimension relative to either mid-shaft portion; and wherein the guidewire has a length and stiffness for each mid-shaft portion that enables the guidewire to be reversibly inserted, with one or the other opposing tip portion leading the way, into a predetermined body passage of a patient. In the foregoing embodiment, the opposing tip portions may have different stiffness and different lengths.

These and other embodiments are described in more detail in the following detailed descriptions and the figures.

The foregoing is not intended to be an exhaustive list of embodiments and features of the inventive subject matter. Persons skilled in the art are capable of appreciating other embodiments and features from the following detailed description in conjunction with the drawings.

### BRIEF DESCRIPTION OF THE DRAWINGS

The following figures show various embodiments of inventive subject matter (except where prior art is noted).

FIG. 1 illustrates a side view of a guidewire.

FIG. 2 is a cross-sectional view of the guidewire shown in FIG. 1, taken along Line A-A.

FIG. 3 is a cross-sectional view of a transition zone of the guidewire shown in FIG. 1, taken along the line B-B.

FIG. 4 is a cross-sectional view of the guidewire shown in FIG. 1, taken along the line C-C.

FIGS. 5A-C show possible cross-sectional views of a guidewire.

FIG. 6 shows a schematic view of a guidewire as it is placed in a straight body passage.

FIG. 7 shows a schematic view of guidewire as it is placed in a tortuous body passage.

### DETAILED DESCRIPTION

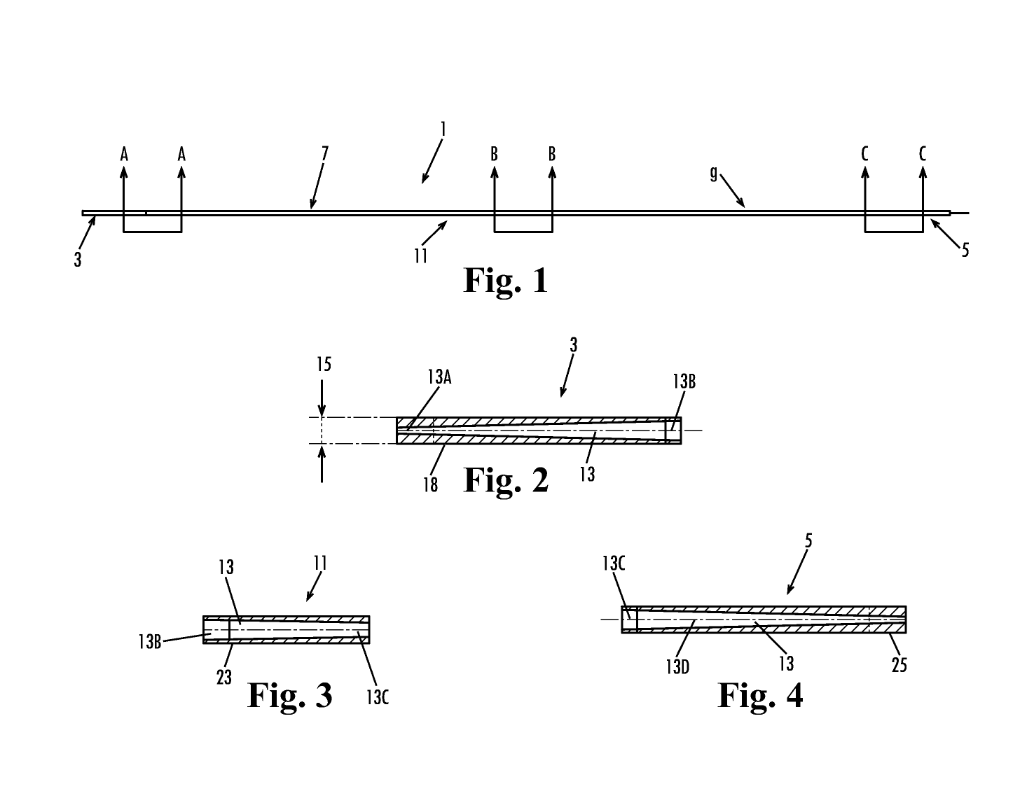

Representative embodiments according to the inventive subject matter are shown in FIGS. 1-7, wherein the same or similar features share common reference numerals.

The inventive subject matter relates to novel reversible, dual floppy guidewires for a medical device or instrument and methods for using such guidewires. As used herein “device” and “instrument” are used interchangeably for any object used to treat, diagnose, inspect, or manipulate a target area of a patient, or to introduce something to the target area. The inventive guidewires are particularly suitable for urology procedures, and for providing a path for endoscopes, access sheaths, stents, catheters and dilators to follow. Guidewires are also used in many other procedures in which an endoscope or other instrument needs to be introduced into a lumen. Common examples include vascular and cardiac procedures, thoracoscopic (lung) procedures, neurological procedures and ENT procedures such as sinus surgery.

In general respects, the guidewire includes opposing tip portions 3, 5 and two or more mid-shaft portions 7, 9 therebetween, which are adjacent each other and the tip portions 3, 5. Each tip portion is relatively less stiff than the mid-shaft portions. The outer cross-sectional profile dimension may be generally uniform across the length of mid-shaft portions. The opposing tip portions may have about the same or smaller cross-sectional profile dimension relative to either mid-shaft portion. The guidewire may further have a length and/or stiffness for each mid-shaft portion 7, 9 that enables the guidewire to be reversibly inserted to deal with varying conditions in a patient, with one or the other opposing tip portion leading the way, into a predetermined body passage of a patient. The guidewire may further have opposing tip portions 3, 5, which relative to one another have different stiffnesses and/or different lengths.

A representative embodiment is illustrated in FIGS. 1-4, and 6-7. Here, a guidewire 1 includes opposing tip portions, 3 and 5, and two mid-shaft portions, 7 and 9, adjacent each other and the tip portions. The two mid-shaft portions, 7 and 9, are interposed between the tip portions, 3 and 5. Each of two mid-shaft portions may start at about the middle of the wire. The tip portions, 3 and 5, are relatively less stiff than either mid-shaft portion, and the mid-shaft portions have a different stiffness relative to each other. Although illustrated at about the midpoint of the wire, the portions may come together anywhere off the midpoint, between the tip portions, so long as each portion has a useful length for an intended use.

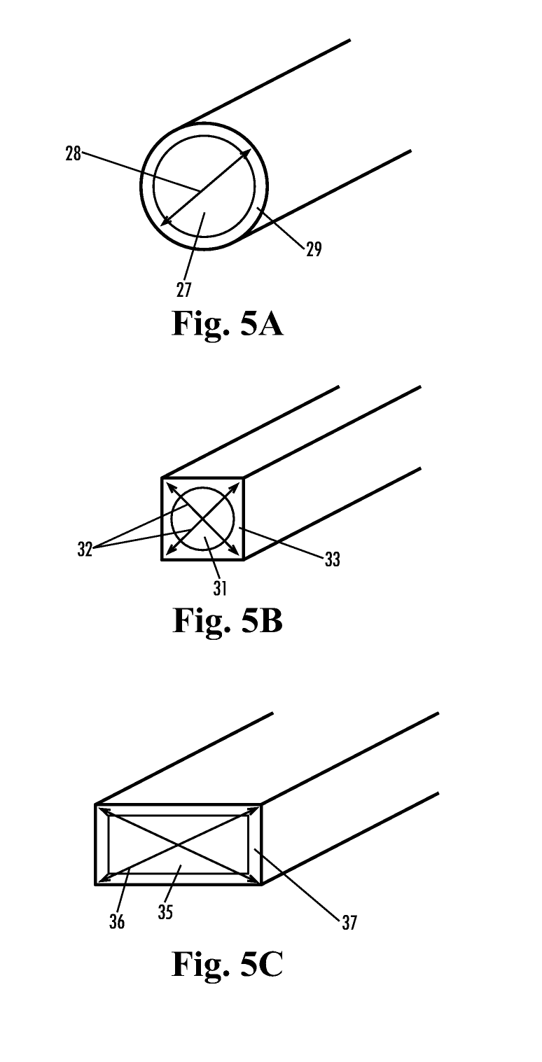

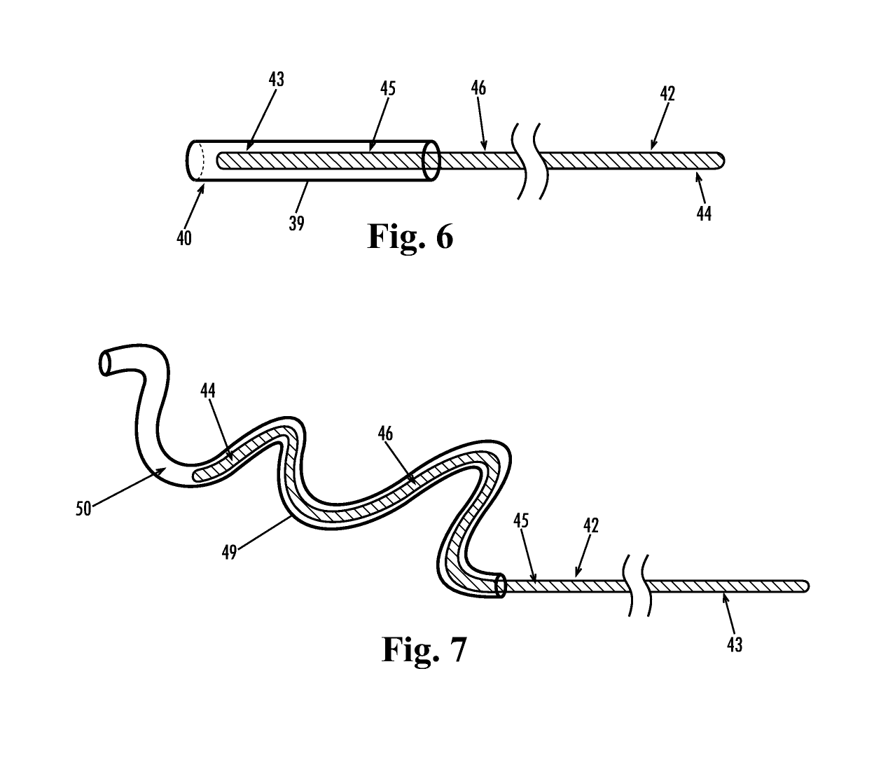

As indicated in FIGS. 1-4, the outer cross-sectional profile dimension 15 of guidewire 1 is uniform across the length of the mid-shaft portions 7 and 9. A uniform, or substantially uniform, diameter facilitates guiding of a device, and use of standard channels on devices for receiving a guidewire. The outer cross-sectional profile dimension is the outer diameter for a circle (FIG. 5A), or other analogous dimension(s) for non-circular shapes (e.g. FIGS. 5B and 5C). The opposing tip portions may have about the same or smaller cross-sectional profile dimension relative to either mid-shaft portion. Different stiffness for different sections of the guidewire may be obtained by variations of inner structure dimensions, as well as by changes in materials or structures.

FIGS. 2-4 illustrate one way for how the stiffness of guidewire 1 may be adjusted while maintaining uniform outside diameter. In the embodiment shown, this is achieved by providing transition zones having different tapered sections of an inner core 13 and complementary, reverse-tapering, surrounding coaxial layer or layers. Core 13 may be formed of a monofilament or multifilament material, as described below.

In this embodiment, core 13 generally provides the basic backbone for placing the guidewire, but has resilience for navigating passages. Guidewire 1 has a substantially uniform cross-sectional profile dimension 15 across the length of mid-shaft portions 7 and 9. The core 13 may have about the same or smaller cross-sectional profile dimension through the opposing tip portions 3 and 5 compared with either mid-shaft portion 7 and 9. The change in the material dimensions of the core therefore varies the stiffness from section to section. To maintain uniform diameter, coaxial material thickness is varied so that the sum of the thickness of the core plus that of the coaxial material is substantially constant along the length of at least the mid-shaft portion.

The coaxial materials may also be placed over the core to selectively stiffen portions of the guidewire. The outer material may also provide low friction surfaces that facilitate movement against tissue. The coaxial material may be one or more layers of polymer materials, metallic webs or jackets, braids, coils etc. Optionally, the outer layer may have a hydrophilic coating. A guidewire, with a low friction coating helps provide for fast, atraumatic access and removal with reduced risk of glide out. The tip coating is highly lubricious, while the mid-shaft portion is relatively less lubricious, thereby allowing the guidewire to navigate obstructions, but reduces the risk of glide out. The opposing two flexible tips help navigate obstructions and tortuous anatomy with little force. A kink resistant core provides enhanced control and easy instrument placement onto the guidewire. Radiopaque tips may be included for accurate placement visualization.

In another embodiment, the core for one mid-shaft portion may be relatively more flexible than the core for the other mid-shaft portion, and the core sections for each tip portion may be about the same flexibility relative to each other.

Optionally, the core itself may have a filamentous structure, including one or more filaments or both. These filaments may run parallel to each other or the filaments may be twisted or otherwise intertwined, for example.

The guidewire 1, shown in FIGS. 1-4, has a filamentous structure based on a central mono-filamentous core 13 surrounded by coaxial layers 18, 23, and 25. The inner core may be made of a shape memory material, such as Nitinol. However, in other embodiments, the core may be made of any shape memory material or super alloy. A shape memory material is a material that can return to some previously defined shape or size when subjected to the appropriate thermal procedure. Generally, “super alloys” are alloys with superior mechanical strength and creep resistance, i.e. resistance to permanent deformation, at high temperatures (above about 700° C.); good surface stability; and corrosion and oxidation resistance. Super alloys typically have a face-centered cubic and austenitic crystal structure. A super alloy's base alloying element is usually nickel, cobalt, or nickel-iron. Examples of super alloys are available from Super Alloys International Limited, Milton Keynes, United Kingdom. (http://www.superalloys.co.uk/products.htm).

Coaxial layers 18, 23, 25 may be made of PTFE (polytetrafluoroethylene) at the mid-shaft portions 7 and 9, and of a lower durometer polymeric coating, such as black PEBAX™ polymer or polyurethane at the floppy tip portions 3 and 5. PEBAX™ is a trademark of Arkema Inc. of Philadelphia, Pa.

In other embodiments, mid-shaft portions 7 and 9 may consist of a core, a first inner coaxial layer including a stainless steel jacket over one or both portions, and an outer coaxial layer consisting of a low friction polymer coating, such as PTFE.

In the embodiment shown in FIGS. 2-4, there is at least some section of each of portion 3, 5 and 7, 9 where the core 13 and surrounding coaxial layers 18, 23, and 25 have complementary tapering to provide transition from a zone of one stiffness to a zone of second stiffness. A transition zone is an area where a first end of a section transitions to a second section having different stiffness. The coaxial layers 18, 23 and 25 surrounding core 13 have a reverse taper complementary to the dimensions of tapered sections of core 13, so that there is an overall cross-sectional profile dimension for the tapering section that is generally uniform. In this embodiment, sections of complementary tapering include opposing tip portions 3 and 5, wherein the core tapers going toward the terminal ends of the tip portions and transition from the mid-shaft portions 7 and 9 to the tip portions 3 and 5 respectively. Another section of complementary taper includes a transition zone 11 between adjacent mid-shaft portions 7 and 9. FIG. 2 shows a tip portion 3 has a uniform outer diameter 15 with a tapered floppy tip wire core 13A, and a complementary reverse tapered coaxial layer 18. The floppy tip wire core 13A gradually tapers into a stiffer core 13B at the mid-shaft portion 7. FIG. 3 illustrates a tapered cross-sectional profile in transition zone 11 between adjacent mid-shaft portions 7 and 9. Here, stiff core 13B gradually tapers into a medium stiffness core 13C. At the transition zone 11, the inner core 13B tapers slightly from a larger diameter at mid-shaft portion 7 to a core 13C with a smaller diameter at mid-shaft portion 9, and is surrounded by coaxial layer 23, having a complementary reverse taper. FIG. 4 shows how medium stiffness core 13C further tapers into floppy tip wire core 13D. Core 13D is surrounded by a complementary reverse tapered coating layer 25.

In an alternative embodiment, the core for a tip portion or mid-shaft portion may have a stepped cross-sectional profile dimension going from one side to another side at at least a transition zone between portions. The corresponding coaxial layer surrounding the core for the stepped section may have a complementary reverse step, so that there is an overall cross-sectional profile dimension for the stepped section that is generally uniform.

The following dimensions are exemplary dimensions of a guidewire for use in ureteroscopy only and may be adjusted by one skilled in the art for other procedures. The embodiment shown in FIGS. 1-4 has a total length of about 150 cm. One opposing tip portion has a different length relative to the other tip portion. Here, the length of floppy tip portion 3 is about 9 cm, while the length of floppy tip portion 5 is about 5 cm. The length of mid-shaft portion 7 is about 68 cm and the length of mid-shaft portion 9 is about 68 cm.

In another embodiment, a guidewire of about 150 cm may include a first floppy tip portion of about 10 cm attached to a first mid-shaft portion of about 65 cm. This first mid-shaft portion may provide for medium stiffness of the guidewire, and may be attached to a second mid-shaft portion providing relatively higher stiffness to the guidewire over a length of about 65 cm. The second floppy tip portion may have a length of about 5 cm.

The guidewire has a uniform outside diameter 15 of 0.1 cm (0.035 inches). Here, the diameter of the core varies over the length of the guidewire and the core shows a tapered profile in different sections. The core 13A of the floppy tip portion 3 is tapered as shown in FIG. 2. The dimensions are about 0.045 cm (0.018 inches) at the outside end section gradually tapering into a diameter of 0.172 cm (0.068 inches) at the opposing end section, i.e., the end of the tip portion where the tip portion attaches to the mid-shaft portion. The core of the mid-shaft portion remains constant with a diameter of about 0.172 cm (0.068 inches), up to transition zone 11, shown in FIG. 3, where the core diameter tapers to a diameter of about 0.146 cm (0.0578 inches) at mid-shaft portion 9. The core diameter of mid-shaft portion 9 remains constant up to floppy tip portion 5, where the core diameter tapers off to about 0.045 cm (0.018 inches) at the end.

The inner cross-sectional profile of a guidewire according to the inventive subject matter may have different dimensions and shapes, while the outer cross-sectional profile dimension is uniform across the length of the guidewire. For example, FIG. 5A shows a cross-sectional circular central core 27 surrounded by an outer cross-sectional circular coaxial layer 29, having a diameter 28. FIG. 5B illustrates a cross-sectional circular core 31, surrounded by a coaxial layer 33 having the shape of a square in cross-section, and determined by diagonal 32. FIG. 5C illustrates a cross-sectional rectangular core 35 surrounded by a coaxial layer 37 having a cross-sectional rectangular shape with a diagonal 36. It is understood by one skilled in the art that any cross-sectional shape or combination of cross-sectional shapes may be used to obtain the desired properties. Additionally, any number of coaxial layers may be used.

In some embodiments, the core of a first mid-shaft portion may have a different cross-sectional profile dimension than the core of a second mid-shaft portion. In other possible embodiments, the thickness of the coaxial layer or layers surrounding the core in a first mid-shaft portion may be different from the thickness of the coaxial layer or layers surrounding the core in a second mid-shaft portion.

In certain embodiments, the opposing tip portions may have end sections comprising a lower durometer polymer coating than the adjacent mid-shaft portions. Other embodiments may have a mid-shaft portion including a core, a first inner coaxial layer comprising a stainless steel jacket, and outer coaxial layer comprising a polymer coating.

In some urologic procedures, in addition to the stiffness of the guidewire being modified, the diameter of the guidewire could be such that a 0.035 inches guidewire has the feel of a 0.038 inches guidewire, for example. The primary reason that physicians prefer a 0.038 inches guidewire is for additional stiffness and support. If the diameter of the entire guidewire was 0.035 inches but it had one end with the stiffness of a 0.035 inches guidewire and one end with the stiffness of a 0.038 inches guidewire, then the design would have greater versatility than existing guidewires and increased flow of irrigation around the guidewire as compared to existing 0.038 inches guidewires. The inventive subject matter further contemplates a method of using a guidewire by providing a reversible guidewire for use in a procedure conducted on a patient. First, the guidewire is inserted into a bodily passage of the patient with one of the opposing tip portions leading the way. Then, the guidewire is withdrawn and reinserted with the other opposing tip portion leading the way. The withdrawal or reinsertion may occur in connection with the placement or removal of a medical instrument over the guidewire, and the instrument's introduction to or from the bodily passage of the patient, in the same treatment episode of treatment.

The inventive subject matter allows the physician to have the performance of two different guidewires in a single design, depending on how the guidewire is inserted into the patient. If the guidewire is inserted one way and the physician needs different performance characteristics, then the physician can simply remove the guidewire and re-insert it with the opposite end entering the patient first. This inventive subject matter will generally avoid the necessity of a physician from having to use two guidewires for one application, and will reduce the amount of inventory that the hospital has to carry.

FIGS. 6 and 7 illustrate how a reversible guidewire may be used in a body passage of a patient. The specific length and stiffness for each mid-shaft portion allow the guidewire to be reversibly inserted. Depending on the shape of the body passage, different combinations of pushability and steerability are desirable. FIG. 6 shows a relatively straight body passage 39, with a target zone 40. The guidewire 42 is inserted in the body passage with a floppy tip portion 43 leading the way and directed towards target zone 40. Mid-shaft portion 45 is partially inserted in the body passage, while mid-shaft portion 46 and floppy tip portion 44 remain outside the patient. FIG. 7 illustrates how the same guidewire may be used during a procedure in which a physician may also need to approach a target zone 50 in a tortuous or convoluted body passage 49. Here, floppy tip portion 44 of guidewire 42 is directed towards target zone 50. Such a convoluted body passage 49 requires more flexibility of mid-shaft portion 46 and floppy tip portion 44, while mid-shaft portion 45 and tip portion 43 provide the right amount of pushability by being less flexible.

Persons skilled in the art will recognize that many modifications and variations are possible in the details, materials, and arrangements of the parts and actions which have been described and illustrated in order to explain the nature of this inventive subject matter and that such modifications and variations do not depart from the spirit and scope of the teachings and claims contained therein.

All patent and non-patent literature cited herein is hereby incorporated by references in its entirety for all purposes.

1. A guidewire for a medical device, comprising:
a filamentous structure having a central core comprising a shape memory material and one or more surrounding coaxial layers;
the structure having opposing tip portions and at least two mid-shaft portions interposed between the tip portions, the tip portions each being relatively less stiff than either mid-shaft portion, and the mid-shaft portions having a different stiffness relative to each other;
the outer cross-sectional profile dimension being generally uniform across the length of mid-shaft portions, the opposing tip portions having about the same or smaller cross-sectional profile dimension relative to either mid-shaft portion; and
wherein the guidewire has a length and stiffness for each mid-shaft portion that enables the guidewire to be reversibly inserted, with one or the other opposing tip portion leading the way, into a predetermined body passage of a patient.

2. The guidewire of claim 1, wherein the core has a substantially uniform cross-sectional profile dimension across the length of mid-shaft portions, and the core through the opposing tip portions having about the same or smaller cross-sectional profile dimension as the core for either mid-shaft portion.

3. The guidewire of claim 1, wherein the core of a first mid-shaft portion has a different cross-sectional profile dimension than the core of a second mid-shaft portion.

4. The guidewire of claim 1, wherein the thickness of the coaxial layer or layers surrounding the core in a first mid-shaft portion is different from the thickness of the coaxial layer or layers surrounding the core in a second mid-shaft portion.

5. The guidewire of claim 3, wherein the core for a tip portion or mid-shaft portion has a tapering cross-sectional profile dimension going from one side to another side at least a transition zone between portions.

6. The guidewire of claim 5, wherein the corresponding coaxial layer surrounding the core for the tapering section has a complementary reverse taper, so that there is an overall cross-sectional profile dimension for the tapering section that is generally uniform.

7. The guidewire of claim 6, wherein there are a plurality of complementary tapering sections, each with a different stiffness.

8. The guidewire of claim 7, wherein at least one section of complementary tapering comprises one opposing tip portion, and another section, the other opposing tip portion, and the core tapers going toward the terminal ends of the tip portions.

9. The guidewire of claim 8, wherein another section of complementary taper comprises a mid-shaft portion.

10. The guidewire of claim 3, wherein the core for a tip portion or mid-shaft portion has a stepped cross-sectional profile dimension going from one side to another side at least a transition zone between portions.

11. The guidewire of claim 10, wherein the corresponding coaxial layer surrounding the core for the stepped section has a complementary reverse step, so that there is an overall cross-sectional profile dimension for the stepped section that is generally uniform.

12. The guidewire of claim 1, wherein the coaxial layer or layers comprise one or more polymer materials.

13. The guidewire of claim 12, wherein the polymer material comprises PTFE.

14. The guidewire of claim 12, wherein the opposing tip portions have end sections comprising a lower durometer polymer coating than the adjacent mid-shaft portions.

15. The guidewire of claim 12, wherein one opposing tip portion has a different length relative to the other tip portion.

16. A guidewire for a medical device, comprising:
a filamentous structure having a central core comprising a super alloy and one or more surrounding coaxial layers;
the structure having opposing tip portions and two mid-shaft portions adjacent each other and the tip portions, the tip portions each being relatively less stiff than either mid-shaft portion;
the outer cross-sectional profile dimension being generally uniform across the length of mid-shaft portions, the opposing tip portions having about the same or smaller cross-sectional profile dimension relative to either mid-shaft portion;
wherein the core for one mid-shaft portion is relatively more flexible than the core for the other mid-shaft portion, and the core sections for each tip portion being about the same flexibility relative to each other; and
wherein the guidewire has a length and stiffness for each mid-shaft portion that enables the guidewire to be reversibly inserted, with one or the other opposing tip portion leading the way, into a predetermined body passage of a patient.

17. The guidewire of claim 1, wherein a mid-shaft portion comprises a core, a first inner coaxial layer comprising a stainless steel jacket, and outer coaxial layer comprising a polymer coating.

18. A method of using a guidewire, comprising;
providing a reversible guidewire for use in a procedure conducted on a patient;
the guidewire comprising:
opposing tip portions and two mid-shaft portions adjacent each other and the tip portions, the tip portions each being relatively less stiff than either mid-shaft portion;
the outer cross-sectional profile dimension being generally uniform across the length of mid-shaft portions, the opposing tip portions having about the same or smaller cross-sectional profile dimension relative to either mid-shaft portion; and
wherein the guidewire has a length and stiffness for each mid-shaft portion that enables the guidewire to be reversibly inserted, with one or the other opposing tip portion leading the way, into a predetermined body passage of a patient;
inserting the guidewire into a bodily passage of the patient with one of the opposing tip portions leading the way;
withdrawing the guidewire and reinserting it with the other opposing tip portion leading the way; and
the withdrawal or reinsertion occurring in connection with the placement or removal of a medical instrument over the guidewire, and the instrument's introduction to or from the bodily passage of the patient, in the same treatment episode of treatment.

19. The method of claim 18, wherein the guidewire comprises:
a filamentous structure having a central core comprising a shape memory material and one or more surrounding coaxial layers;
the structure having opposing tip portions and at least two mid-shaft portions interposed between the tip portions, the tip portions each being relatively less stiff than either mid-shaft portion, and the mid-shaft portions having a different stiffness relative to each other;
the outer cross-sectional profile dimension being generally uniform across the length of mid-shaft portions, the opposing tip portions having about the same or smaller cross-sectional profile dimension relative to either mid-shaft portion; and
wherein the guidewire has a length and stiffness for each mid-shaft portion that enables the guidewire to be reversibly inserted, with one or the other opposing tip portion leading the way, into a predetermined body passage of a patient.

20. The method of claim 18, wherein the guidewire comprises:
a filamentous structure having a central core comprising a super alloy and one or more surrounding coaxial layers;
the structure having opposing tip portions and two mid-shaft portions adjacent each other and the tip portions, the tip portions each being relatively less stiff than either mid-shaft portion;
the outer cross-sectional profile dimension being generally uniform across the length of mid-shaft portions, the opposing tip portions having about the same or smaller cross-sectional profile dimension relative to either mid-shaft portion;
wherein the core for one mid-shaft portion is relatively more flexible than the core for the other mid-shaft portion, and the core sections for each tip portion being about the same flexibility relative to each other; and
wherein the guidewire has a length and stiffness for each mid-shaft portion that enables the guidewire to be reversibly inserted, with one or the other opposing tip portion leading the way, into a predetermined body passage of a patient.

21. A guidewire for a medical device, comprising:
opposing tip portions and two mid-shaft portions adjacent each other and the tip portions, the tip portions each being relatively less stiff than either mid-shaft portion;
the outer cross-sectional profile dimension being generally uniform across the length of mid-shaft portions, the opposing tip portions having about the same or smaller cross-sectional profile dimension relative to either mid-shaft portion; and
wherein the guidewire has a length and stiffness for each mid-shaft portion that enables the guidewire to be reversibly inserted, with one or the other opposing tip portion leading the way, into a predetermined body passage of a patient.

22. The guidewire of claim 21, wherein the opposing tip portions have different stiffness.

23. The guidewire of claim 21, wherein the opposing tip portions have different lengths.
